(19) United States
(12) Patent Application Publication
(10) Pub. No.: US 2026/0141995 A1
(43) Pub. Date: May 21, 2026

(54) INTELLIGENT HEALTHCARE FEEDBACK SURVEYS

(71) Applicant: MatrixCare, Inc., Bloomington, MN (US)

(72) Inventor: Elizabeth Boyd SILVA, Burlington, MA (US)

(21) Appl. No.: 19/394,170

(22) Filed: Nov. 19, 2025

Related U.S. Application Data
(60) Provisional application No. 63/723,243, filed on Nov. 21, 2024.

(51) Int. Cl. G16H 10/20; G16H 10/60; G16H 50/20

(52) U.S. Cl. CPC G16H 10/20; G16H 10/60; G16H 50/20

(57) ABSTRACT

Techniques for improved machine learning are provided. Electronic survey questions relating to patient care at a healthcare facility are determined. The electronic survey questions are disseminated to one or more recipients based on a survey trigger, comprising transmitting the electronic survey questions to electronic devices associated with each of the recipients. One or more treatment recommendations are generated for patient care at the healthcare facility, comprising: receiving, from the electronic devices associated with each of the recipients, responses to the electronic survey questions, and inferring the one or more treatment recommendations by providing the responses to a trained machine learning (ML) model. The one or more treatment recommendations are provided to the healthcare facility, where the one or more treatment recommendations are used to improve patient care at the healthcare facility.

### CROSS-REFERENCE TO RELATED APPLICATIONS

This application claims benefit of co-pending U.S. provisional patent application Ser. No. 63/723,243 filed Nov. 21, 2024. The aforementioned related patent application is herein incorporated by reference in its entirety.

### INTRODUCTION

Aspects of the present disclosure relate to artificial intelligence and healthcare, and more specifically, to intelligent healthcare feedback surveys using machine learning (ML).

Healthcare providers (e.g., home healthcare and hospice providers) are often frustrated by the lack of timely feedback available, and the limited ability to capture feedback from patients and caregivers. This limited feedback makes it very difficult for providers to identify issues (e.g., during a patient's stay with the provider) and very difficult to address those issues. For example, existing surveys for hospice care (e.g., hospice consumer assessment of healthcare providers and systems (CAHPS) surveys) typically do not provide survey results until many months after a patient has passed away.

### SUMMARY

According to some embodiments of the present disclosure, a method is provided. The method includes determining a plurality of electronic survey questions relating to patient care at a healthcare facility; disseminating the plurality of electronic survey questions to one or more recipients based on a survey trigger, comprising: transmitting the plurality electronic survey questions to an electronic device associated with each of the one or more recipients; generating one or more treatment recommendations for patient care at the healthcare facility, comprising: receiving, from the respective electronic device associated with each of the one or more recipients, a plurality of responses to the plurality of electronic survey questions; and inferring the one or more treatment recommendations by providing the plurality of responses to a trained machine learning (ML) model; and providing the one or more treatment recommendations to the healthcare facility, wherein the one or more treatment recommendations are used to improve patient care at the healthcare facility.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts a computing environment for intelligent healthcare feedback surveys, according to one embodiment.

FIG. 2 depicts a block diagram for a prediction controller for intelligent healthcare feedback surveys, according to one embodiment.

FIG. 3 is a flowchart illustrating intelligent healthcare feedback surveys, according to one embodiment.

FIG. 4 is a flowchart illustrating training a survey generation ML model for intelligent healthcare feedback surveys, according to one embodiment.

FIG. 5 is a flowchart illustrating generating intelligent healthcare feedback survey questions using an ML model, according to one embodiment.

FIG. 6 is a flowchart illustrating training a survey analysis ML model for intelligent healthcare feedback surveys, according to one embodiment.

FIG. 7 is a flowchart illustrating inferring treatment recommendations from responses to intelligent healthcare feedback survey questions, using an ML model, according to one embodiment.

FIG. 8 is a flowchart illustrating treatment recommendations for intelligent healthcare feedback surveys, according to one embodiment.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

As discussed above, existing systems provide very limited feedback for healthcare providers. One or more embodiments discussed herein provide for techniques to capture rapid (e.g., real-time) feedback from patients, caregivers, and others, while a patient is currently in a healthcare program and receiving services. In an embodiment, one or more surveys (e.g., mini-surveys made up of a relatively small number of survey questions) can be generated and administered to recipients electronically (e.g., via short message service (SMS) message, e-mail, or any other suitable electronic message). These surveys can be administered frequently (e.g., nightly) and can provide rapid feedback from recipients. Recipients can include patients, caregivers (e.g., care providers and other healthcare employees), family members of patients, and any other suitable recipients. The feedback from the surveys can be captured quickly, and used to provide treatment recommendations for a patient or healthcare facility.

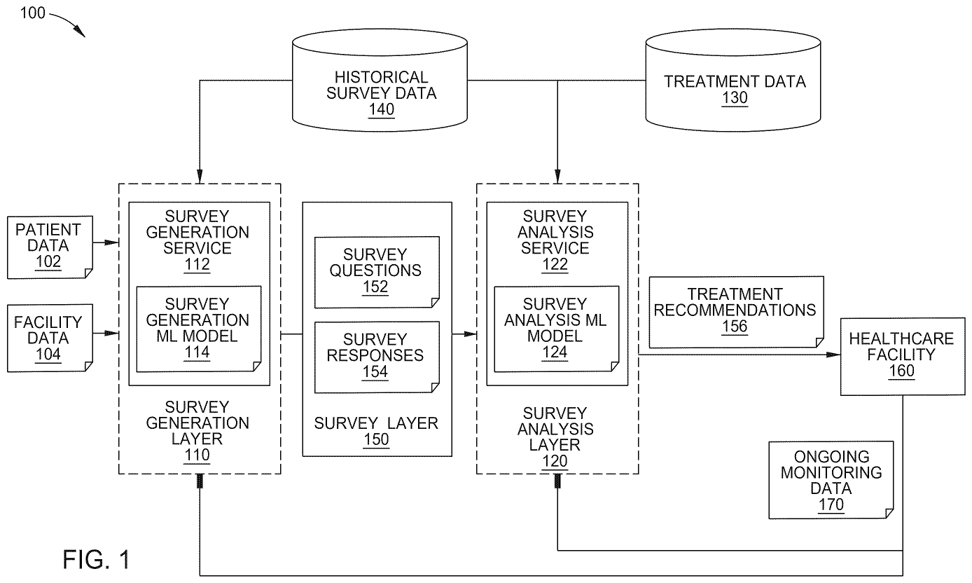

Further, in an embodiment, ML can be leveraged to generate these rapid surveys, infer treatment recommendations from the survey results, or both. For example, a suitable ML model can be used to automatically (e.g., without human intervention) generate survey questions based on patient data, facility data, historical survey data, and any other suitable input data. These survey questions can be distributed to recipients, electronically, and responses can be gathered. The survey responses can then be analyzed by an additional ML model, which can infer treatment recommendations based on the survey responses and suitable additional data (e.g., treatment data, historical survey data, or any other suitable data). This is discussed further, below, with regard to FIG. 1.

In an embodiment, surveys can be triggered at a variety of time points. For example, a survey could be generated about the healthcare facility admission process, shortly after a patient has been admitted. Similarly, a survey could be generated about family interactions with caregivers, shortly after a family meeting with caregivers. These surveys can be provided to, and gather data from, patients, patient family members, care providers, and other healthcare employees, among other recipients. This allows for a wide variety of feedback and can generate a wide variety of useful treatment recommendations.

Thus, aspects described herein provide significant advantages compared to conventional approaches for generating healthcare surveys. For example, inferring treatment recommendations automatically, using a trained ML model, based on frequent survey responses provides for accurate improvements to patient treatment outcomes while minimizing the needed computational resources for the prediction and shifting the computational burden from prediction time (e.g., when near real-time response may be needed) to an earlier training time (e.g., when resources can be easily dedicated to the training). In an embodiment, analyzing survey responses using a specific rubric or algorithm with pre-defined rules can be computationally expensive, because a very large number of rules are needed and parsing and following the rules is computationally expensive. Further, this computationally expensive analysis is done at the time the treatment recommendation is generated, when a rapid response is likely to be needed (e.g., so that the treatment recommendations can be implemented quickly).

Inferring treatment recommendations for survey responses automatically using a trained ML model, by contrast, is significantly less computationally expensive at the time the treatment recommendations are generated. For example, the prediction ML model can be trained up-front during a training phase, when rapid response is not necessary and computational resources are readily available. The trained ML model can then be used to rapidly, and computationally relatively cheaply, predict treatment recommendations for the patient. This provides a significant technical advantage over prior techniques by shifting the computational burden from the prediction time, when a rapid response is needed and computational resources may be engaged in other tasks, to a planned training time when a rapid response is not necessary and computational resources are available.

As another example, inferring treatment recommendations, generating survey questions, or both, automatically using a trained ML model provides for a more accurate and well-defined prediction. In an embodiment, survey questions could be manually drafted by a care provider, and survey responses could be manually reviewed by a care provider. But this leaves the risk of human error and allows for significant variances among human practitioners, which can result in a lack of certainty in the accuracy of the treatment recommendations. Predicting the survey questions, treatment recommendations, or both, using a trained ML model can both lessen the risk of human error, and provide more certainty in the level of accuracy of the treatment recommendations. Further, the predicted treatment recommendations and survey questions can themselves be reviewed and refined by a care provider or other reviewer. This provides a starting point for the reviewer with a more certain level of accuracy, and reduces the burden on the reviewer.

FIG. 1 depicts a computing environment 100 for intelligent healthcare feedback surveys, according to one embodiment. In an embodiment, input data is provided to a survey generation layer 110. For example, patient data 102 (e.g., patient demographic data, patient medical history data, or any other suitable patient data) and facility data 104 (e.g., facility characteristics, facility employee data, facility equipment data, or any other suitable facility data) can be provided to the survey generation layer 110. Further, historical survey data 140 (e.g., data reflecting prior survey questions, answers, response rates, other statistical data, and any other suitable historical survey data) can be provided to the survey generation layer 110. For example, the historical survey data 140 can include a bank of survey questions, along with benchmarking for responses to these questions. These are merely examples, and any suitable data can be provided to the survey generation layer 110.

In an embodiment, the input data (e.g., any, or all, of the patient data 102, facility data 104, and historical survey data 140) is provided to the survey generation layer 110 using a suitable communication network. This can include any suitable communication network, including the Internet, a wide area network, a local area network, or a cellular network, and can use any suitable wired or wireless communication technique (e.g., WiFi or cellular communication). This is merely one example, and the input data can be provided to the survey generation layer 110 using any suitable technique (e.g., using storage medium or through a direct wired or wireless transmission).

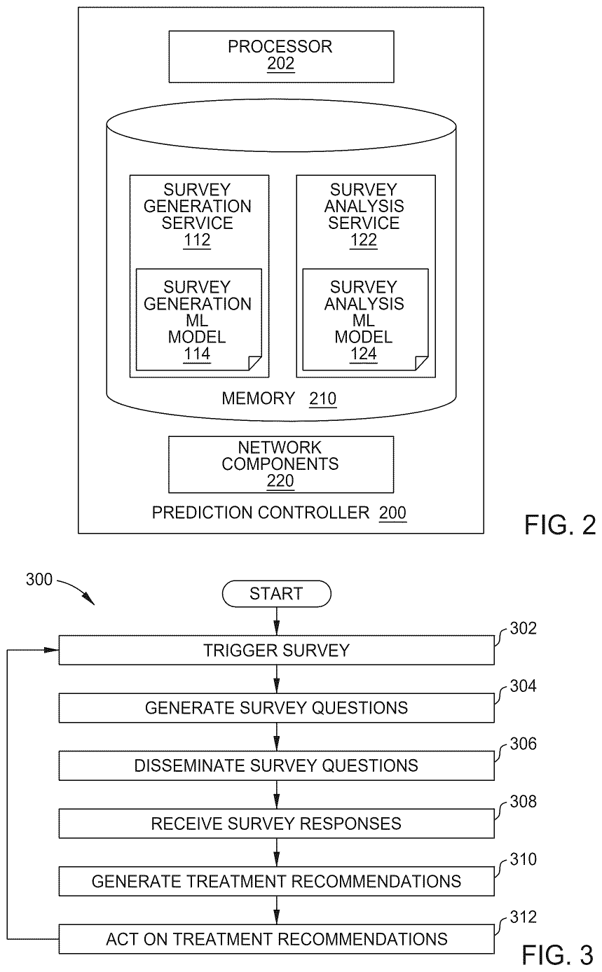

The survey generation layer 110 includes a survey generation service 112, which includes a survey generation ML model 114. In an embodiment, the survey generation 112 facilitates generating one or more healthcare surveys (e.g., one or more survey questions) using the input data. For example, as discussed below with regard to FIG. 2, the survey generation service 112 can be computer software service implemented in a suitable controller (e.g., the prediction controller 200 illustrated in FIG. 2) or combination of controllers. In an embodiment the survey generation 110, and the survey generation service 112, can be implemented using any suitable combination of physical compute systems, cloud compute nodes and storage locations, or any other suitable implementation. For example, the survey generation 110 could be implemented using a server or cluster of servers. As another example, the survey generation 110 can be implemented using a combination of compute nodes and storage locations in a suitable cloud environment. For example, one or more of the components of the survey generation 110 can be implemented using a public cloud, a private cloud, a hybrid cloud, or any other suitable implementation.

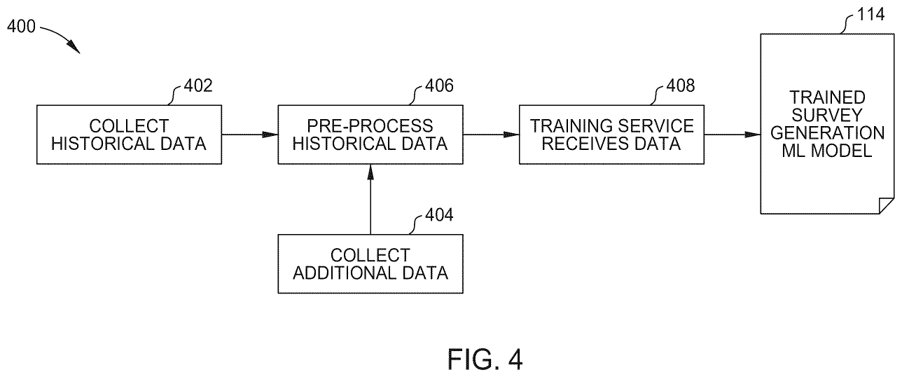

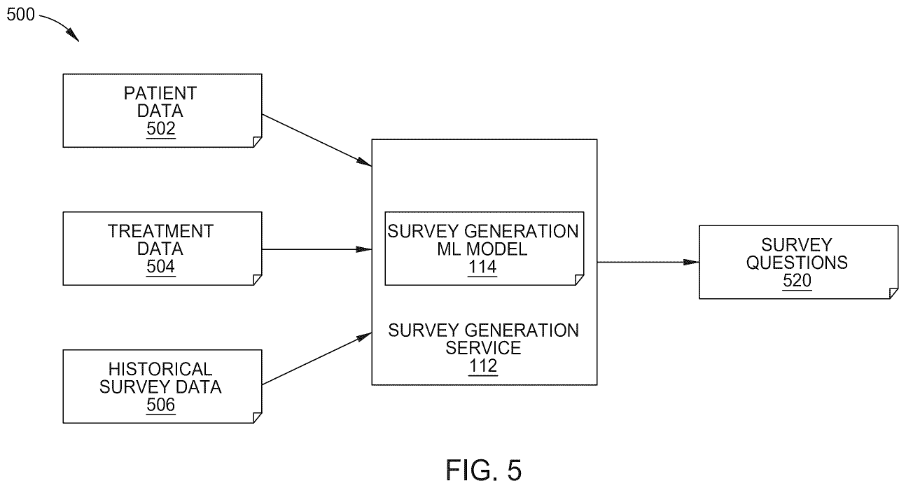

As one example, the survey generation service can facilitate use of the survey generation ML model (e.g., a large language model (LLM) or any other suitable ML model) to generate one or more survey questions 152 using the input data (e.g., any suitable combination of the patient data 102, facility data 104, historical survey data 140, and any other suitable data). In this example, the survey generation service can be a deep learning neural network trained to generate the survey questions 152. This is merely an example, and the survey generation service 112 can use any suitable ML model. This is discussed further, below, with regard to FIGS. 4-5.

In an embodiment, the survey generation ML model 114 can include multiple ML models trained to generate survey questions from input data. For example, different ML models could be trained to generate survey questions for different aspects of a healthcare environment (e.g., different points in time, different categories of patients, different categories of facilities (e.g., in-patient and out-patient facilities). In some aspects, these different models may be used ensemble to produce a prediction. This is merely an example.

Further, in an embodiment the survey generation service 112 can generate the survey questions 152 without using ML. For example, the survey generation service 112 can identify the survey questions 152 from a question bank of previously generated survey questions (e.g., as part of the historical survey data 140). The question bank can further be associated with benchmarking for responses, which can be combined with a rules-based approach to generate survey questions. These are merely examples, and the survey generation service 112 can use any suitable technique.

In an embodiment, the survey generation layer 110 generates the survey questions 152. A survey layer 150 can distribute these survey questions to recipients (e.g., care providers, other healthcare workers, patients, family members of patients, or any other suitable recipients) and can gather survey responses 154 (e.g., responses to the survey questions 152). The survey layer 150 can provide the survey responses 154 to a survey analysis layer 120. In an embodiment, the survey responses 154 are numerical responses, narrative responses, or any suitable combination.

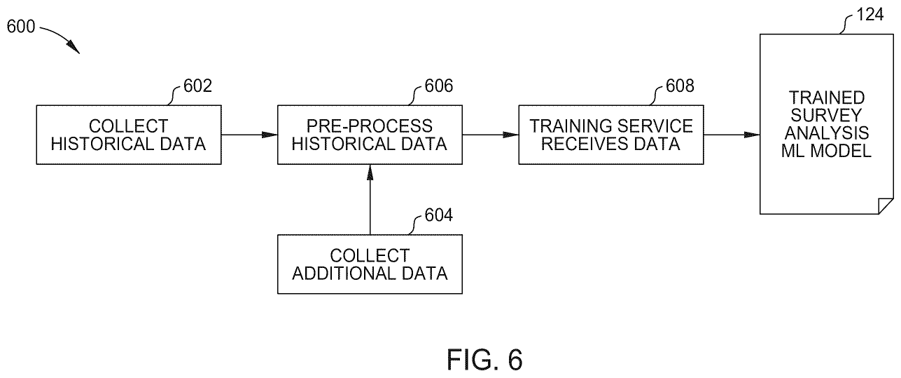

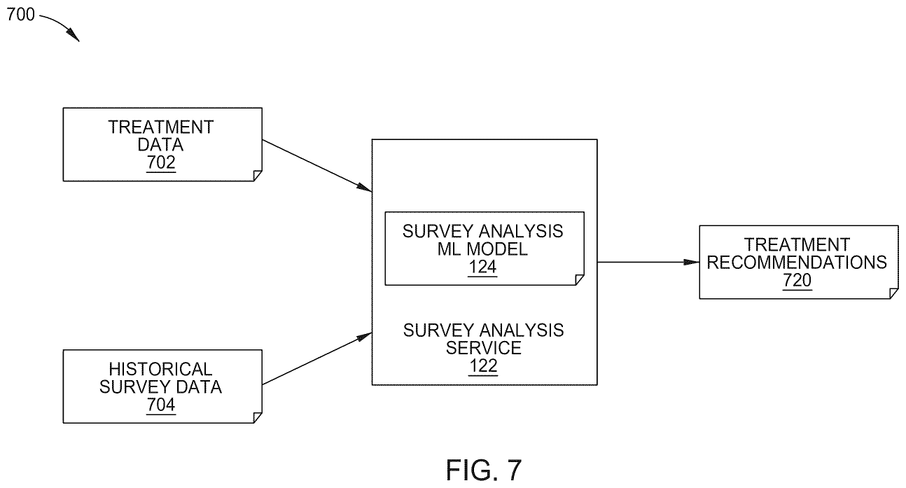

The survey analysis layer 120 includes a survey analysis service 122 and a survey analysis ML model 124. In an embodiment, the survey analysis service 122 facilitates analysis of survey responses 154 to infer treatment recommendations 156. As discussed above, the treatment recommendations 156 can include recommended changes, policies, procedures, actions, or any other suitable items to improve treatment outcomes for patients. For example, the survey analysis service 122 can use the survey analysis ML model 124 to infer the treatment recommendations 156. This is discussed further below with regard to FIGS. 6-7. In an embodiment, suitable natural language processing (NLP) techniques (e.g., a suitable ML model) can be used to parse and analyze narrative survey responses 154 (e.g., prior to providing the responses to the survey analysis ML model 124). As another example, suitable statistical techniques can be used to analyze numerical survey responses 154 (e.g., prior to providing the responses to the survey analysis ML model 124).

For example, the treatment recommendations 156 can include care planning information, improvements for clinical resource assignments (e.g., identifying a staffing shortage), risk mitigation (e.g., identifying potential fall predictions or wound management issues), improvements for patient assignments (e.g., for in-home care vs in-facility care, or to select among healthcare facilities), improvements for patient communication (e.g., tailored communication channels and techniques for patients), improvements for patient or caregiver anxiety (e.g., identifying potential anxiety issues and providing recommendations to change staffing or other aspects to improve the issues), or any other suitable treatment recommendations.

In an embodiment, the treatment recommendations 156 can further be used to provide for an immediate alert to a facility or care provider, for a given patient. For example, the treatment recommendations 156 could indicate an urgent issue, and the survey analysis service 122 could contact the healthcare facility or care provider (e.g., using an SMS message, automated phone call, or any other suitable electronic notification) to provide prophylactic treatment to assist the patient.

As discussed below with regard to FIG. 2, the survey analysis service 122 can be computer software service implemented in a suitable controller (e.g., the prediction controller 200 illustrated in FIG. 2) or combination of controllers. In an embodiment the survey analysis 120, and the survey analysis service 122, can be implemented using any suitable combination of physical compute systems, cloud compute nodes and storage locations, or any other suitable implementation. For example, the survey analysis 120 could be implemented using a server or cluster of servers. As another example, the survey analysis layer 120 can be implemented using a combination of compute nodes and storage locations in a suitable cloud environment. For example, one or more of the components of the survey analysis layer 120 can be implemented using a public cloud, a private cloud, a hybrid cloud, or any other suitable implementation.

As discussed above, the survey analysis layer 120 uses the survey responses 154 to infer the treatment recommendations 156. In an embodiment, however, the survey responses 154 are not sufficient to allow the survey analysis layer 120 to accurately infer the treatment recommendations 156. For example, merely receiving the survey responses 154 may not provide necessary context or details need to infer suitable treatment recommendations 156.

In an embodiment, the survey analysis layer 120 can further receive, and use, treatment data 130 (e.g., facility data, patient data, treatment data, or any other suitable data) and historical survey data 140. For example, the treatment data 130 can include facility data (e.g., characteristics of one or more healthcare facilities relating to the survey questions 152), patient data (e.g., characteristics of patients relating to the survey questions 152), and any other suitable data. As discussed above, in an embodiment the historical survey data 140 can include historical survey questions, responses, analysis, and any other suitable historical survey data. In an embodiment, the treatment data 130 and historical survey data 140 has had any personally identifying patient information removed.

In an embodiment, the treatment data 130 and the historical survey data 140 are provided to the survey analysis layer 120 using a suitable communication network. For example, the treatment data 130 and the historical survey data 140 can be stored in one or more suitable electronic databases (e.g., a relational database, a graph database, or any other suitable database) or other electronic repositories (e.g., a cloud storage location, an on-premises network storage location, or any other suitable electronic repository). The treatment data 130 and the historical survey data 140 can be provided from the respective electronic repositories to the survey analysis layer 120 using any suitable communication network, including the Internet, a wide area network, a local area network, or a cellular network, and can use any suitable wired or wireless communication technique (e.g., WiFi or cellular communication).

As discussed above, in an embodiment, the survey analysis service 122 uses the analysis ML model 124 to infer treatment recommendations 156 based on the survey responses 154. For example, the analysis ML model 124 can be a suitable supervised ML model (e.g., a DNN) trained to generate one or more treatment recommendations 156 from a combination of survey responses, treatment data 130, and historical survey data 140. This is discussed further below with regard to FIGS. 6-7. For example, the analysis ML model 124 can be selected based on initial analysis of the characteristics of the input data (e.g., the survey responses 154, treatment data 130, and historical survey data 140). In an embodiment, a basic technique can be initially selected (e.g., logistic regression), data can be converted to a numerical format, and based on initial analysis data transformation and ML techniques can be chosen. This is merely an example, and any suitable supervised, or unsupervised, techniques can be used.

For example, the survey analysis ML model 124 can predict treatment recommendations 156. In an embodiment, the treatment recommendations 156 can be provided to a healthcare facility 160. In an embodiment, the healthcare facility 160 can be any suitable in-patient or out-patient treatment facility, including a hospice facility, an in-home healthcare facility, or any other suitable healthcare facility. Further, in an embodiment, the treatment recommendations 156 can be provided directly to the patient or to the patient's medical care provider.

In an embodiment, the treatment recommendations 156 is used to improve patient treatment for the healthcare facility 160. For example, the treatment recommendations 156 can include recommended policy changes, infrastructure changes, staffing changes, or any other suitable recommendations for the healthcare facility 160. In an embodiment, treatment outcomes at the healthcare facility can be monitored, and ongoing monitoring data 170 can be gathered. For example, statistical data, progress notes, additional survey data, and any other suitable data can be gathered.

In an embodiment, this ongoing patient monitoring data 170 can be provided to the survey generation layer 110, the survey analysis layer 120, or both, and used to refine the survey questions 152, treatment recommendations 156, or both. For example, the ongoing monitoring data 170 can be used to supplement, or replace, the facility data 104 used by the survey generation layer 110, the treatment data 130 used by the survey analysis layer 120, or both.

FIG. 2 depicts a block diagram for a prediction controller 200 for intelligent healthcare feedback surveys, according to one embodiment. The controller 200 includes a processor 202, a memory 210, and network components 220. The memory 210 may take the form of any non-transitory computer-readable medium. The processor 202 generally retrieves and executes programming instructions stored in the memory 210. The processor 202 is representative of a single central processing unit (CPU), multiple CPUs, a single CPU having multiple processing cores, graphics processing units (GPUs) having multiple execution paths, and the like.

The network components 220 include the components necessary for the controller 200 to interface with a suitable communication network (e.g., a communication network interconnecting various components of the computing environment 100 illustrated in FIG. 1, or interconnecting the computing environment 100 with other computing systems). For example, the network components 220 can include wired, WiFi, or cellular network interface components and associated software. Although the memory 210 is shown as a single entity, the memory 210 may include one or more memory devices having blocks of memory associated with physical addresses, such as random access memory (RAM), read only memory (ROM), flash memory, or other types of volatile and/or non-volatile memory.

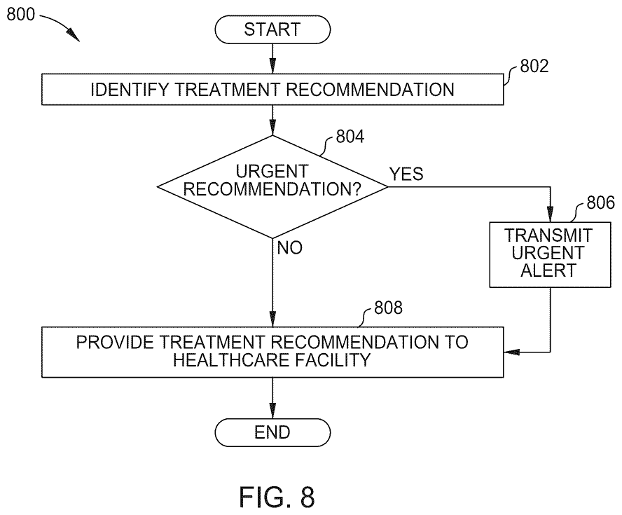

The memory 210 generally includes program code for performing various functions related to use of the prediction controller 200. The program code is generally described as various functional “applications” or “modules” within the memory 210, although alternate implementations may have different functions and/or combinations of functions. Within the memory 210, the survey generation service 112 facilitates generating survey questions using the survey generation ML model 114. This is discussed further below with regard to FIGS. 4-6. The survey analysis service 122 facilitates predicting treatment recommendations, using the survey analysis ML model 124. This is discussed further below with regard to FIGS. 7-8.

While the controller 200 is illustrated as a single entity, in an embodiment, the various components can be implemented using any suitable combination of physical compute systems, cloud compute nodes and storage locations, or any other suitable implementation. For example, the controller 200 could be implemented using a server or cluster of servers. As another example, the controller 200 can be implemented using a combination of compute nodes and storage locations in a suitable cloud environment. For example, one or more of the components of the controller 200 can be implemented using a public cloud, a private cloud, a hybrid cloud, or any other suitable implementation.

Although FIG. 2 depicts the survey generation service 112, the survey analysis service 122, the survey generation ML model 114, and the survey analysis ML model 124, as being mutually co-located in memory 210, that representation is also merely provided as an illustration for clarity. More generally, the controller 200 may include one or more computing platforms, such as computer servers for example, which may be co-located, or may form an interactively linked but distributed system, such as a cloud-based system, for instance. As a result, processor 202 and memory 210 may correspond to distributed processor and memory resources within the computing environment 100. Thus, it is to be understood that any, or all, of survey generation service 112, the survey analysis service 122, the survey generation ML model 114, and the survey analysis ML model 124 may be stored remotely from one another within the distributed memory resources of the computing environment 100.

FIG. 3 is a flowchart 300 illustrating intelligent healthcare feedback surveys, according to one embodiment. At block 302 a survey generation service (e.g., the survey generation service 112 illustrated in FIGS. 1-2) triggers a survey. In an embodiment, the survey generation service can trigger the survey based on a time period, events, treatment outcomes, or any other suitable trigger(s). For example, as discussed above surveys can be provided frequently to capture real-time (or near real-time) responses from recipients. This can include intermittent, consistent, surveys (e.g., nightly, weekly, or at any other suitable interval), surveys following designated events (e.g., intake, family meetings, staff meetings, treatments, procedures, or any other suitable events), or surveys at any other suitable time.

At block 304 the survey generation service generates survey questions. For example, as illustrated in FIG. 1, the survey generation service can receive any combination of patient data 102, facility data 104, historical survey data 140, and any other suitable data. The survey generation service can use these inputs to generate survey questions. For example, the survey generation service can use a survey generation ML model (e.g., the survey generation ML model 114 illustrated in FIGS. 1-2) to infer one or more survey questions. This is discussed further, below, with regard to FIGS. 4-5. Alternatively, or in addition, the survey generation service can generate the survey questions without using ML. For example, the survey generation service 112 can identify survey questions from a repository of previously generated survey questions, using a rules-based approach, or using any other suitable technique

At block 306 the survey generation service disseminates the survey questions. In an embodiment, the survey generation service transmits the survey questions electronically to electronic devices (e.g., smartphones, tablets, wearable devices, desktop computers, laptop computers, or any other suitable electronic devices) for suitable recipients, including patients, patient family members, care providers, other healthcare workers, or any other suitable recipients. For example, the survey generation service can transmit the survey electronically using a web interface, mobile or desktop App, electronic mail, SMS message, other electronic message, automated phone call, or using any other suitable technique.

At block 308, a survey analysis service (e.g., the survey analysis service 122 illustrated in FIGS. 1-2) receives survey responses. In an embodiment, as discussed above in relation to FIG. 1, the survey responses are gathered electronically. The survey responses can then be used to generate treatment recommendations (e.g., using a suitable ML model).

At block 310, the survey analysis service generates treatment recommendations. For example, as discussed above in relation to FIG. 1, the survey analysis service can use a suitable ML model (e.g., the survey analysis ML model 124 illustrated in FIG. 1) to generate treatment recommendations. The treatment recommendations can include recommended changes, policies, procedures, actions, or any other suitable items to improve treatment outcomes for patients. For example, the survey analysis service can use the survey analysis ML model to infer the treatment recommendations. This is discussed further below with regard to FIGS. 6-7.

At block 312, a recipient acts on treatment recommendations. For example, as discussed above in relation to FIG. 1, the treatment recommendations can include care planning information, improvements for clinical resource assignments (e.g., identifying a staffing shortage), risk mitigation (e.g., identifying potential fall predictions or wound management issues), improvements for patient assignments (e.g., for in-home care vs in-facility care, or to select among healthcare facilities), improvements for patient communication (e.g., tailored communication channels and techniques for patients), improvements for patient or caregiver anxiety (e.g., identifying potential anxiety issues and providing recommendations to change staffing or other aspects to improve the issues), or any other suitable treatment recommendations. In an embodiment, the treatment recommendations can further be used to provide for an immediate alert to a facility or care provider, for a given patient. Acting on the treatment recommendations is discussed further, below, with regard to FIG. 8.

FIG. 4 is a flowchart 400 illustrating training a survey generation ML model for intelligent healthcare feedback surveys, according to one embodiment. This is merely an example, and in an embodiment a suitable unsupervised technique could be used (e.g., without requiring training). At block 402, a training service (e.g., a human administrator or a software or hardware service) collects historical data (e.g., historical patient data, historical treatment data, historical survey data, or any other suitable historical data). For example, a survey generation service (e.g., the survey generation service 112 illustrated in FIGS. 1-2) can be configured to act as a training service, and can collect historical data reflecting patient characteristics, treatment characteristics and outcomes, survey questions, responses, and statistical analysis, and any other suitable historical data (e.g., gathered over time).

At block 406, the training service (or other suitable service) pre-processes the collected historical data. For example, the training service can create feature vectors reflecting the values of various features, for each prior survey, patient group, treatment, care provider, facility, or any other suitable delineation. At block 408, the training service receives the feature vectors and uses them to train a trained survey generation ML model 114.

In an embodiment, at block 404 the training service also collects additional data. For example, the training service can use additional historical language data to assist in training the trained survey generation ML model 114.

At block 406, the training service can also pre-process this additional data. For example, the feature vectors corresponding to the historical data collected at block 402 can be further annotated using the additional data. Alternatively, or in addition, additional feature vectors corresponding to the additional data can be created. At block 408, the training service uses the pre-processed additional data during training to generate the trained survey generation ML model 114.

In an embodiment, the pre-processing and training can be done as batch training. In this embodiment, all data is pre-processed at once, and provided to the training service at block 408. Alternatively, the pre-processing and training can be done in a streaming manner. In this embodiment, the data is streaming, and is continuously pre-processed and provided to the training service. For example, it can be desirable to take a streaming approach for scalability. The set of training data may be very large, so it may be desirable to pre-process the data, and provide it to the training service, in a streaming manner (e.g., to avoid computation and storage limitations). Further, in an embodiment, a federated learning approach could be used in which multiple entities contribute to training a shared model.

FIG. 5 is a flowchart 500 illustrating generating intelligent healthcare feedback survey questions using an ML model, according to one embodiment. A survey generation service 112 (e.g., as illustrated in FIGS. 1-2) is associated with a survey generation ML model 114. In an embodiment, the survey generation ML model 114 is trained to predict one or more survey questions 520. This is discussed above in relation to FIG. 3. For example, the survey generation ML model 114 can predict one or more survey questions to disseminate to recipients..

In an embodiment, the survey generation service 112 uses multiple types of data to predict survey questions 520. For example, the survey generation service 112 can use patient data 502 (e.g., patient data 102 illustrated in FIG. 1), treatment data 504 (e.g., treatment data 104 illustrated in FIG. 1), historical survey data 506 (e.g., historical survey data 140 illustrated in FIG. 1), or any other suitable data. The survey generation service 112 can use the survey generation ML model 114 to infer (e.g., predict) the survey questions 520.

In an embodiment, each of the one or more survey questions 520 identifies a single best match for a survey question. Alternatively, or in addition, the survey questions 520, identify multiple suggested matches. In an embodiment, a care provider or other user can the select a preferred option among the multiple suggested matches, one or more rules could be used to select among options, or any other suitable technique can be used.

FIG. 6 is a flowchart 600 illustrating training a survey analysis ML model for intelligent healthcare feedback surveys, according to one embodiment. This is merely an example, and in an embodiment a suitable unsupervised technique could be used (e.g., without requiring training). At block 602, a training service (e.g., a human administrator or a software or hardware service) collects historical data (e.g., historical patient data, historical treatment data, historical survey data, or any other suitable historical data). For example, a survey analysis service (e.g., the survey analysis service 122 illustrated in FIGS. 1-2) can be configured to act as a training service, and can collect historical data reflecting patient characteristics, treatment characteristics and outcomes, survey questions, responses, and statistical analysis, and any other suitable historical data (e.g., gathered over time).

At block 606, the training service (or other suitable service) pre-processes the collected historical data. For example, the training service can create feature vectors reflecting the values of various features, for each prior survey, patient group, treatment, care provider, facility, or any other suitable delineation. At block 608, the training service receives the feature vectors and uses them to train a trained survey analysis ML model 124.

In an embodiment, at block 604 the training service also collects additional data. For example, the training service can use additional historical language data to assist in training the trained survey analysis ML model 124.

At block 606, the training service can also pre-process this additional data. For example, the feature vectors corresponding to the historical data collected at block 602 can be further annotated using the additional data. Alternatively, or in addition, additional feature vectors corresponding to the additional data can be created. At block 608, the training service uses the pre-processed additional data during training to generate the trained survey analysis ML model 114.

In an embodiment, the pre-processing and training can be done as batch training. In this embodiment, all data is pre-processed at once, and provided to the training service at block 608. Alternatively, the pre-processing and training can be done in a streaming manner. In this embodiment, the data is streaming, and is continuously pre-processed and provided to the training service. For example, it can be desirable to take a streaming approach for scalability. The set of training data may be very large, so it may be desirable to pre-process the data, and provide it to the training service, in a streaming manner (e.g., to avoid computation and storage limitations). Further, in an embodiment, a federated learning approach could be used in which multiple entities contribute to training a shared model.

FIG. 7 is a flowchart 700 illustrating inferring treatment recommendations from responses to intelligent healthcare feedback survey questions, using an ML model, according to one embodiment. A survey analysis service 122 (e.g., as illustrated in FIGS. 1-2) is associated with a survey analysis ML model 124. In an embodiment, the survey analysis ML model 124 is trained to predict one or more treatment recommendations 720. This is discussed above in relation to FIG. 3. For example, the survey analysis ML model 124 can predict one or more treatment recommendations to disseminate to healthcare facilities, care providers, or any other suitable recipients.

In an embodiment, the survey analysis service 122 uses multiple types of data to predict the treatment recommendations 720. For example, the survey analysis service 122 can use treatment data 702 (e.g., treatment data 130 illustrated in FIG. 1), historical survey data 704 (e.g., historical survey data 140 illustrated in FIG. 1), or any other suitable data. The survey analysis service 122 can use the survey analysis ML model 12 to infer (e.g., predict) the treatment recommendations 720.

In an embodiment, each of the one or more treatment recommendations 720 provides a single best match. Alternatively, or in addition, the treatment recommendations 720, identify multiple suggested matches. In an embodiment, a care provider or other user can the select a preferred option among the multiple suggested matches, one or more rules could be used to select among options, or any other suitable technique can be used.

FIG. 8 is a flowchart 800 illustrating treatment recommendations for intelligent healthcare feedback surveys, according to one embodiment. At block 802 a survey analysis service (e.g., the survey analysis service 122 illustrated in FIG. 1) identifies a treatment recommendation. For example, as discussed above in relation to FIG. 1, the treatment recommendations can include care planning information, improvements for clinical resource assignments (e.g., identifying a staffing shortage), risk mitigation (e.g., identifying potential fall predictions or wound management issues), improvements for patient assignments (e.g., for in-home care vs in-facility care, or to select among healthcare facilities), improvements for patient communication (e.g., tailored communication channels and techniques for patients), improvements for patient or caregiver anxiety (e.g., identifying potential anxiety issues and providing recommendations to change staffing or other aspects to improve the issues), or any other suitable treatment recommendations.

At block 804, the survey analysis service determines whether the treatment recommendation is an urgent recommendation. As discussed above, a treatment recommendation can further be used to provide for an immediate alert to a facility or care provider, for a given patient. The survey analysis service can analyze the treatment recommendation by identifying an associated code signifying that the treatment recommendation is urgent, using NLP techniques to parse a textual description and identify the treatment recommendation as urgent, or using any other suitable technique.

If the survey analysis service identifies the treatment recommendation as urgent, the flow proceeds to block 806. At block 806, the survey analysis service transmits an urgent alert. For example, the survey analysis service could contact the healthcare facility or care provider (e.g., using an SMS message, automated phone call, or any other suitable electronic notification) to identify the urgent alert. The recipient can then use the alert to provide prophylactic treatment to assist the patient.

At block 808, the survey analysis service provides treatment recommendations to the healthcare facility (e.g., non-urgent recommendations). In an embodiment, as discussed above in relation to FIG. 1, the survey analysis service transmits the treatment recommendations to a relevant healthcare facility (e.g., the healthcare facility 160 illustrated in FIG. 1). The recipient can then use the recommendations to improve patient care. A healthcare facility is merely one example of a suitable recipient, and the survey analysis service can provide the treatment recommendations to any suitable recipient.

In an embodiment, the recipient of the treatment recommendations (e.g., a healthcare facility) automatically takes action to improve treatment without human intervention. For example, the healthcare facility could change scheduling (e.g., patient or staff scheduling) based on the treatment recommendation, automatically generate an alert or recommendation to change staff activities, or take any other suitable action. Alternatively, or in addition, the treatment recommendations are provided for human review, and the human reviewer enacts changes to improve treatment outcomes.

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method, comprising:
determining a plurality of electronic survey questions relating to patient care at a healthcare facility;
disseminating the plurality of electronic survey questions to one or more recipients based on a survey trigger, comprising:
transmitting the plurality electronic survey questions to an electronic device associated with each of the one or more recipients;
generating one or more treatment recommendations for patient care at the healthcare facility, comprising:
receiving, from the respective electronic device associated with each of the one or more recipients, a plurality of responses to the plurality of electronic survey questions; and
inferring the one or more treatment recommendations by providing the plurality of responses to a trained machine learning (ML) model; and
providing the one or more treatment recommendations to the healthcare facility, wherein the one or more treatment recommendations are used to improve patient care at the healthcare facility.

2. The method of claim 1, wherein the healthcare facility comprises at least one of: (i) a hospice facility or (ii) a home healthcare facility.

3. The method of claim 2, wherein the survey trigger comprises an event at the healthcare facility.

4. The method of claim 1, wherein inferring the one or more treatment recommendations by providing the plurality of responses to the trained ML model further comprises:
providing to the trained ML model treatment data reflecting characteristics of patient care at the healthcare facility, in addition to the plurality of responses.

5. The method of claim 4, wherein determining the plurality of electronic survey questions relating to patient care at a healthcare facility comprises:
generating the plurality of electronic survey questions using a second trained ML model.

6. The method of claim 5, wherein generating the plurality of electronic survey questions using a second trained ML model comprises:
providing to the second trained ML model historical survey data and data relating to patient care at the healthcare facility.

7. The method of claim 1, further comprising:
identifying ongoing monitoring data for the healthcare facility, captured after providing the one or more treatment recommendations to the healthcare facility; and
generating a second one or more treatment recommendations for patient care at the healthcare facility based on both: (i) a second plurality of responses to a second plurality of electronic survey questions, and (ii) the ongoing monitoring data.

8. The method of claim 1, further comprising:
determining that a first treatment recommendation of the one or more treatment recommendations comprises an urgent recommendation; and
generating an alert for a care provider at the healthcare facility, based on the first treatment recommendation, wherein the alert is used by the care provider to provide prophylactic treatment to a patient.

9. The method of claim 1,
wherein the one or more treatment recommendations are provided to the healthcare facility electronically, and
wherein the one or more treatment recommendations are used to automatically, without human intervention, modify patient care at the healthcare facility.

10. A non-transitory computer program product comprising:
one or more non-transitory computer readable media containing, in any combination, computer program code that, when executed by operation of any combination of one or more processors, performs operations comprising:
determining a plurality of electronic survey questions relating to patient care at a healthcare facility;
disseminating the plurality of electronic survey questions to one or more recipients based on a survey trigger, comprising:
transmitting the plurality electronic survey questions to an electronic device associated with each of the one or more recipients;
generating one or more treatment recommendations for patient care at the healthcare facility, comprising:
receiving, from the respective electronic device associated with each of the one or more recipients, a plurality of responses to the plurality of electronic survey questions; and
inferring the one or more treatment recommendations by providing the plurality of responses to a trained machine learning (ML) model; and
providing the one or more treatment recommendations to the healthcare facility, wherein the one or more treatment recommendations are used to improve patient care at the healthcare facility.

11. The non-transitory computer program product of claim 10, wherein the healthcare facility comprises at least one of: (i) a hospice facility or (ii) a home healthcare facility, and wherein the survey trigger comprises an event at the healthcare facility.

12. The non-transitory computer program product of claim 10, wherein inferring the one or more treatment recommendations by providing the plurality of responses to the trained ML model further comprises:
providing to the trained ML model treatment data reflecting characteristics of patient care at the healthcare facility, in addition to the plurality of responses.

13. The non-transitory computer program product of claim 12, wherein determining the plurality of electronic survey questions relating to patient care at a healthcare facility comprises:
generating the plurality of electronic survey questions using a second trained ML model.

14. The non-transitory computer program product of claim 10, further comprising:
identifying ongoing monitoring data for the healthcare facility, captured after providing the one or more treatment recommendations to the healthcare facility; and
generating a second one or more treatment recommendations for patient care at the healthcare facility based on both: (i) a second plurality of responses to a second plurality of electronic survey questions, and (ii) the ongoing monitoring data.

15. The non-transitory computer program product of claim 10, further comprising:
determining that a first treatment recommendation of the one or more treatment recommendations comprises an urgent recommendation; and
generating an alert for a care provider at the healthcare facility, based on the first treatment recommendation, wherein the alert is used by the care provider to provide prophylactic treatment to a patient.

16. A system, comprising:
one or more processors; and
one or more memories storing a program, which, when executed on any combination of the one or more processors, performs operations, the operations comprising:
determining a plurality of electronic survey questions relating to patient care at a healthcare facility;
disseminating the plurality of electronic survey questions to one or more recipients based on a survey trigger, comprising:
transmitting the plurality electronic survey questions to an electronic device associated with each of the one or more recipients;
generating one or more treatment recommendations for patient care at the healthcare facility, comprising:
receiving, from the respective electronic device associated with each of the one or more recipients, a plurality of responses to the plurality of electronic survey questions; and
inferring the one or more treatment recommendations by providing the plurality of responses to a trained machine learning (ML) model; and
providing the one or more treatment recommendations to the healthcare facility, wherein the one or more treatment recommendations are used to improve patient care at the healthcare facility.

17. The system of claim 16, wherein the healthcare facility comprises at least one of: (i) a hospice facility or (ii) a home healthcare facility, and wherein the survey trigger comprises an event at the healthcare facility.

18. The system of claim 16, wherein inferring the one or more treatment recommendations by providing the plurality of responses to the trained ML model further comprises:
providing to the trained ML model treatment data reflecting characteristics of patient care at the healthcare facility, in addition to the plurality of responses.

19. The system of claim 18, wherein determining the plurality of electronic survey questions relating to patient care at a healthcare facility comprises:
generating the plurality of electronic survey questions using a second trained ML model.

20. The system of claim 16, further comprising:
determining that a first treatment recommendation of the one or more treatment recommendations comprises an urgent recommendation; and
generating an alert for a care provider at the healthcare facility, based on the first treatment recommendation, wherein the alert is used by the care provider to provide prophylactic treatment to a patient.
